SEQUENCE LISTING (16 sequences)

SEQ ID NO: 1 - protein, 419 aa, homosapiens
ANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 2 - protein, 461 aa, homosapiens
MWQLTSLLLFVATWGISGTPAPLDSVFSSSERAHQVLRIRKRANSFLEELRHSSLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 3 - protein, 461 aa, homosapiens
MWQLTSLLLFVATWGISGTPAPLDSVFSSSERAHQVLRIRKRANSFLEELRHSHLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 4 - protein, 461 aa, homosapiens
MWQLTSLLLFVATWGISGTPAPLDSVFSSSERAHQVLRIRKRANSFLEELRHSKLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 5 - protein, 461 aa, homosapiens
MWQLTSLLLFVATWGISGTPAPLDSVFSSSERAHQVLRIRKRANSFLEELRHSNLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 6 - protein, 461 aa, homosapiens
MWQLTSLLLFVATWGISGTPAPLDSVFSSSERAHQVLRIRKRANSFLEELRHGKLERECIEEICDFEEAKEIFQNVDDTLAFWSKHVDGDQCLVLPLEHPCASLCCGHGTCIDGIGSFSCDCRSGWEGRFCQREVNFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 7 - protein, 461 aa, homosapiens
MWQLTSLLLFVATWGISGTPAPLDSVFSSSERAHQVLRIRKRANSFLEELRQGKLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 8 - protein, 461 aa, homosapiens
MWQLTSLLLFVATWGISGTPAPLDSVFSSSERAHQVLRIRKRANSFLEELRQGNLERECIEEICDFEEAKEIFQNVDDTLAFWSKHVDGDQCLVLPLEHPCASLCCGHGTCIDGIGSFSCDCRSGWEGRFCQREVSFLNCSLDNGGCTHYCLEEVGWRRCSCAPGYKLGDDLLQCHPAVKFPCGRPWKRMEKKRSHLKRDTEDQEDQVDPRLIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 9 - DNA, 1260 bp, homosapiens
gccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 10 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcagcctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 11 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagccacctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 12 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcaagctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 13 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacagcaacctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 14 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcacggcaagctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgaacttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 15 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcaagggaaactggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

SEQ ID NO: 16 - DNA, 1386 bp, homosapiens
atgtggcagctcacaagcctcctgctgttcgtggccacctggggaatttccggcacaccagctcctcttgactcagtgttctccagcagcgagcgtgcccaccaggtgctgcggatccgcaaacgtgccaactccttcctggaggagctccgtcaagggaacctggagcgggagtgcatagaggagatctgtgacttcgaggaggccaaggaaattttccaaaatgtggatgacacactggccttctggtccaagcacgtcgacggtgaccagtgcttggtcttgcccttggagcacccgtgcgccagcctgtgctgcgggcacggcacgtgcatcgacggcatcggcagcttcagctgcgactgccgcagcggctgggagggccgcttctgccagcgcgaggtgagcttcctcaattgctcgctggacaacggcggctgcacgcattactgcctagaggaggtgggctggcggcgctgtagctgtgcgcctggctacaagctgggggacgacctcctgcagtgtcaccccgcagtgaagttcccttgtgggaggccctggaagcggatggagaagaagcgcagtcacctgaaacgagacacagaagaccaagaagaccaagtagatccgcggctcattgatgggaagatgaccaggcggggagacagcccctggcaggtggtcctgctggactcaaagaagaagctggcctgcggggcagtgctcatccacccctcctgggtgctgacagcggcccactgcatggatgagtccaagaagctccttgtcaggcttggagagtatgacctgcggcgctgggagaagtgggagctggacctggacatcaaggaggtcttcgtccaccccaactacagcaagagcaccaccgacaatgacatcgcactgctgcacctggcccagcccgccaccctctcgcagaccatagtgcccatctgcctcccggacagcggccttgcagagcgcgagctcaatcaggccggccaggagaccctcgtgacgggctggggctaccacagcagccgagagaaggaggccaagagaaaccgcaccttcgtcctcaacttcatcaagattcccgtggtcccgcacaatgagtgcagcgaggtcatgagcaacatggtgtctgagaacatgctgtgtgcgggcatcctcggggaccggcaggatgcctgcgagggcgacagtggggggcccatggtcgcctccttccacggcacctggttcctggtgggcctggtgagctggggtgagggctgtgggctccttcacaactacggcgtttacaccaaagtcagccgctacctcgactggatccatgggcacatcagagacaaggaagccccccagaagagctgggcaccttag

(19) United States
(12) Patent Application Publication
(10) Pub. No.: US 2006/0204489 A1
(43) Pub. Date: Sep. 14, 2006

(54) Protein C derivatives

(76) Inventors: Bruce Edward Gerlitz, Indianapolis, IN (US); Brian William Grinnell, Indianapolis, IN (US); Lihua Huang, Carmel, IN (US); Bryan Edward Jones, Carmel, IN (US)

Correspondence Address: ELI LILLY & COMPANY, PATENT DIVISION, P.O. BOX 6288, INDIANAPOLIS, IN, 46206-6288, US

(21) Appl. No.: 11/397,046

(22) Filed: Apr. 3, 2006

Related U.S. Application Data
(63) Continuation of application No. 10/129,893, filed on May 9, 2002, now abandoned.
(60) Provisional application No. 60/166,623, filed on Nov. 19, 1999.

(51) Int. Cl. A61K 38/48; C12N 9/64

(52) U.S. Cl. 424/94.63; 530/325; 435/226

(57) ABSTRACT

Novel human protein C derivatives are described. These derivatives have increased anti-coagulation activity compared to wild-type protein C and retain the biological activity of the wild-type human protein C. These derivatives will require either less frequent administration and/or smaller dosage than wild-type human protein C in the treatment of acute coronary syndromes, vascular occlusive disorders, hypercoagulable states, thrombotic disorders and disease states predisposing to thrombosis.

### PRIORITY

This application is a continuation of a co-pending U.S. application Ser. No. 10/129,893 filed May 9, 2002 which claimed the benefit of U.S. provisional application Ser. No. 60/166,623 filed Nov. 19, 1999.

### FIELD OF THE INVENTION

This invention relates to novel polynucleotides, polypeptides encoded by them and to the use of such polynucleotides and polypeptides. More specifically, the invention relates to human protein C derivatives with increased anti-coagulant activity as compared to wild type activated protein C, to their production, and to pharmaceutical compositions comprising these human protein C derivatives.

### BACKGROUND OF THE INVENTION

Protein C is a serine protease and naturally occurring anti-coagulant that plays a role in the regulation of hemostasis by inactivating Factors Vₐ and VIIIₐ in the coagulation cascade. Human protein C is made in vivo as a single polypeptide of 461 amino acids. This polypeptide undergoes multiple post-translational modifications including, 1) cleavage of a 42 amino acid signal sequence; 2) cleavage of lysine and arginine residues (positions 156 and 157) to make a 2-chain inactive precursor or zymogen (an 155 amino acid residue light chain attached via a disulfide bridge to a 262 amino acid residue heavy chain); 3) vitamin K-dependent carboxylation of nine glutamic acid residues located within the amino-terminal 45 residues (gla-domain); and, 4) carbohydrate attachment at four sites (one in the light chain and three in the heavy chain). Finally, the 2-chain zymogen may be activated by removal of a dodecapeptide at the N-terminus of the heavy chain, producing activated protein C (aPC) possessing greater enzymatic activity than the 2-chain zymogen.

Blood coagulation is a highly complex process regulated by the balance between pro-coagulant and anti-coagulant mechanisms. This balance determines a condition of either normal hemostasis or abnormal pathological thrombus generation and the progression, for example, of coronary thrombosis leading to acute coronary syndromes (ACS; e.g. unstable angina, myocardial infarction). Two major factors control this balance, the generation of fibrin and the activation and subsequent aggregation of platelets, both processes controlled by the generation of the enzyme thrombin, which occurs following activation of the clotting cascade. Thrombin, when bound to thrombomodulin, also functions as a potent anti-coagulant since it activates protein C zymogen to aPC, which in turn inhibits the generation of thrombin. Thus, through the feedback regulation of thrombin generation via the inhibition of Factors Va and VIIIa, aPC functions as perhaps the most important down-regulator of blood coagulation resulting in protection against thrombosis. In addition to anti-coagulation, aPC has anti-inflammatory effects, and exerts profibrinolytic properties that facilitate clot lysis.

Arterial thrombosis occurs in ACS in response to endothelial injury, typically as a result of a disruption of lipid-rich plaque. The initial phases of this response involve platelet adhesion, activation, and assembly of various procoagulants at the site of injury and on the surfaces of activated platelets. The resultant elaboration of thrombin generation plays a critical role in the progression of thrombus formation: both by fibrin deposition, and by platelet activation, thus potentiating the activation of the coagulation system. Traditional (e.g. unfractionated heparin [UFH]) and current (e.g. low-molecular weight heparin [LMWH]) anti-coagulant therapies for ACS rely on the inhibition of thrombin and/or Factor Xa (e.g. the heparins inactivate both thrombin and Xa by dramatically stimulating their interaction with anti-thrombin-III). However, due to steric constraints, these agents are not as effective in inhibiting clot-bound Xa or thrombin. The ability of aPC to target and to irreversibly inactivate the clot-bound Xa/Va complex attenuates local thrombin generation and the progression of thrombosis. Thus, aPC provides an advantage compared to current inhibitors of thrombin or Xa since the effect of decreased thrombin generation will persist after concentrations of aPC have decayed.

The critical role of aPC in controlling hemostasis is also exemplified by the increased rate of thrombosis in heterozygous deficiency, protein C resistance (e.g., due to the common Factor V Leiden mutation) and the fatal outcome of untreated homozygous protein C deficiency. Plasma-derived and recombinantly produced aPC have been shown to be effective and safe antithrombotic agents in a variety of animal models of both venous and arterial thrombosis.

Protein C levels have also been shown to be abnormally low in the following diseases and conditions: disseminated intravascular coagulation (DIC) [Fourrier, et al., Chest 101:816-823, 1992], sepsis [Gerson, et al., Pediatrics 91:418-422, 1993], major trauma/major surgery [Thomas, et al., Am J Surg. 158:491-494, 1989], burns [Lo, et al., Burns 20:186-187 (1994)], adult respiratory distress syndrome (ARDS) [Hasegawa, et al., Chest 105(1):268-277, 1994], and transplantations [Gordon, et al., Bone Marrow Trans. 11:61-65 (1993)]. In addition, there are numerous diseases with thrombotic abnormalities or complications that aPC may be useful in treating, such as: heparin-induced thrombocytopenia (HIT) [Phillips, et al., Annals of Pharmacotherapy 28: 43-45, 1994], sickle cell disease or thalassemia [Karayalcin, et al., The American Journal of Pediatric Hematology/Oncology 11(3):320-323, 1989], viral hemorrhagic fever [Lacy, et al., Advances in Pediatric Infectious Diseases 12:21-53, 1997], thrombotic thrombocytopenic purpura (TTP) and hemolytic uremic syndrome (HUS) [Moake, Seminars in Hematology 34(2):83-89, 1997]. In addition, aPC in combination with Bactericidal Permeability Increasing Protein (BPI) may be useful in the treatment of sepsis [Fisher, et al., Crit. Care Med. 22(4):553-558, 1994].

It is well established that platelet inhibition is efficacious in both prevention and treatment of thrombotic disease. However, the use of anti-platelet agents, such as aspirin, increase the risk of bleeding, which limits the dose of the agent and duration of treatment. The combination of aPC and anti-platelet agents results in a synergy that allows the reduction of the dosages of both aPC and the anti-platelet agent(s). The reduction of the dosages of the agents in combination therapy in turn results in reduced side effects such as increased bleeding often observed in combination anti-coagulant/anti-platelet therapy.

Various methods of obtaining protein C from plasma and producing protein C, aPC and protein C/aPC polypeptides through recombinant DNA technology are known in the art and have been described. See e.g., U.S. Pat. Nos. 4,775,624 and 5,358,932. Despite improvements in methods to produce aPC through recombinant DNA technology, aPC and polypeptides thereof are difficult and costly to produce. Therefore, an aPC derivative exhibiting increased anti-coagulant activity, while maintaining the other biological activities of aPC (e.g., fibrinolytic, and anti-inflammatory activities), provides a compound that is effectively more potent than the parent compound, requiring substantially reduced dosage levels for therapeutic applications.

Enhancement of human protein C calcium and membrane binding activity by site-directed mutagenesis of the gla-domain been reported by several investigators, for example, Shen et al. (J Biol. Chem., 273(47) 31086-91, 1998) and Shen et al. (Biochemistry, 36(51) 16025-31, 1997). Through continued scientific experiments, analysis, and innovation, we identified specific sites and modified targeted amino acid residues in the gla-domain of the aPC molecule. Surprisingly, we found increased anti-coagulant activity of the aPC derivative when specific site-directed mutations were performed. In particular, site specific mutagenesis at amino acid positions: 10 (His), 11 (Ser) and 12 (Ser) of SEQ ID NO: 1, alone or in combinations thereof were found to have increased anti-coagulant activity when compared to wild-type aPC.

Accordingly, the present invention describes novel human protein C derivatives. These human protein C derivatives retain the important biological activity of the wild-type protein C and have substantially greater anti-coagulant activity than wild-type aPC. Therefore, these compounds provide various advantages, e.g. less frequent administration and/or smaller dosages and thus a reduction in the overall cost of production and therapy. Furthermore, these compounds exhibit an advantage over traditional anti-coagulant therapies in disease states, such as, ACS. Importantly, the increases in human protein C derivative anti-coagulant activity may be achieved via one to three amino acid substitutions, which are less likely to be immunogenic in comparison to molecules which contain more than three amino acid substitutions (U.S. Pat. No. 5,358,932; Holly, et al., Biochemistry 33:1876-1880, 1994).

### SUMMARY OF THE INVENTION

The present invention provides a human protein C derivative comprising SEQ ID NO: 1 and the corresponding amino acid in SEQ ID NO: 2, wherein one or more of amino acids at positions 10, 11, or 12 is substituted with an amino acid selected from Ala, Arg, Asn, Asp, Cys, Glu, Gln, Gly, His, Ile, Leu, Lys, Met, Phe, Pro, Thr, Ser, Trp, Tyr, and Val provided that amino acid 10 is not His, amino acid 11 is not Ser, and amino acid 12 is not Ser. The invention further provides the activated form of the above-identified human protein C derivatives.

The present invention also provides recombinant DNA molecules encoding the human protein C derivatives in the preceding paragraph, in particular those comprising SEQ ID NOS: 11, 12, 13, 14, 15, and 16.

Another aspect of the present invention provides protein sequences of these same human protein C derivatives, particularly those comprising SEQ ID NOS: 3, 4, 5, 6, 7, and 8 and the activated forms of these human protein C derivatives.

The present invention comprises methods of treating, acute coronary syndromes such as myocardial infarction and unstable angina.

The present invention further comprises methods of treating thrombotic disorders. Such disorders include, but are not limited to, stroke, abrupt closure following angioplasty or stent placement, and thrombosis as a result of peripheral vascular surgery.

The present invention further comprises methods of treating vascular occlusive disorders and hypercoagulable states including: sepsis, disseminated intravascular coagulation, purpura fulminans, major trauma, major surgery, burns, adult respiratory distress syndrome, transplantations, deep vein thrombosis, heparin-induced thrombocytopenia, sickle cell disease, thalassemia, viral hemorrhagic fever, thrombotic thrombocytopenic purpura, and hemolytic uremic syndrome.

The invention further provides treating the above mentioned diseases and conditions by administering to a patient in need thereof a pharmaceutically effective amount of a human protein C derivative selected from the human protein C derivative comprising SEQ ID NO: 1 and the corresponding amino acid in SEQ ID NO: 2 wherein one or more of amino acids at positions 10, 11 or 12 is substituted with an amino acid selected from Ala, Arg, Asn, Asp, Cys, Glu, Gln, Gly, His, Ile, Leu, Lys, Met, Phe, Pro, Thr, Ser, Trp, Tyr, and Val provided that amino acid 10 is not His, amino acid 11 is not Ser, and amino acid 12 is not Ser. The invention further provides treating these same diseases and conditions employing the activated form of the above-identified human protein C derivatives. Preferred human protein C derivatives include S12K, S12N, S12H, S11G:S12K, H10Q:S11G:S12K, and H10Q:S11G:S12N.

Another embodiment of the present invention is a method of treating sepsis comprising the administration to a patient in need thereof a pharmaceutically effective amount of a human protein C derivative of this invention in combination with bacterial permeability increasing protein. Preferred human protein C derivatives include S12K, S12N, S12H, S11G:S12K, H10Q:S11G:S12K, and H10Q:S11G:S12N.

Another embodiment of the present invention is a method of treating thrombotic disorders which comprises: administering to a patient in need thereof a pharmaceutically effective amount of a human protein C derivative of this invention in combination with an anti-platelet agent. Preferred human protein C derivatives include S12K, S12N, S12H, S11G:S12K, H10Q:S11G:S12K, and H10Q: S11G: S12N.

Another embodiment of the present invention is a method of treating acute arterial thrombotic occlusion, thromboembolism, or stenosis in coronary, cerebral or peripheral arteries or in vascular grafts comprising: administering to a patient in need thereof a pharmaceutically effective amount of an activated human protein C derivative with increased anti-coagulation activity when compared to wild-type activated human protein C, in combination with a thrombolytic agent. Preferred human protein C derivatives include S12K, S12N, S12H, S11G:S12K, H10Q:S11G:S12K, and H10Q:S11G:S12N.

Yet another embodiment of the present invention is a method of treating human patients with genetically predisposed prothrombotic disorders which comprises administering gene therapy to said patients with a recombinant DNA molecule encoding a protein C derivative with increased anti-coagulation activity when compared to wild-type activated human protein C. Preferred human protein C derivatives include S12K, S12N, S12H, S11G:S12K, H10Q:S11G:S12K, and H10Q:S11G:S12N.

Another aspect of the invention comprises treating the diseases and conditions caused or resulting from protein C deficiency as defined herein. This aspect of the invention contemplates any and all modifications to any aPC molecule resulting in increased anti-coagulant activity as compared to wild-type aPC.

Another embodiment of the present invention is a human protein C derivative produced by a process wherein phosphorylation of the serine residue at position 12 of SEQ ID NO: 1 and the corresponding amino acid of SEQ ID NO: 2 is inhibited.

The present invention also provides a pharmaceutical composition comprising, a pharmaceutically acceptable carrier or diluent and a human protein C derivative of this invention, preferably selected from S12K, S12N, S12H, S11G:S12K, H10Q:S11G:S12K, and H10Q:S11G:S12N.

Methods and aspects of producing the novel human protein C derivatives are also an aspect of this invention.

The present invention also provides an article of manufacture for human pharmaceutical use, comprising packaging material and a vial comprising lyophilized human activated protein C derivative with increased anti-coagulation activity when compared to wild-type activated human protein C, wherein said packaging material comprises a label which indicates that said activated protein C be administered by continuous infusion at a dosage of about 0.01 μg/kg/hr to about 50 μg/kg/hr.

### DETAILED DESCRIPTION OF THE INVENTION

For purposes of the present invention, as disclosed and claimed herein, the following terms are as defined below.

Anti-platelet agent—one or more agents alone or in combination which reduces the ability of platelets to aggregate. Agents understood and appreciated in the art include those cited in, for example, Remington, The Science and Practice of Pharmacy, Nineteenth Edition, Vol II, pages 924-25, Mack Publishing Co., herein incorporated by reference. Such agents include but are not limited to aspirin (ASA), clopidogrel, ReoPro® (abciximab), dipyridamole, ticlopidine and IIb/IIIa antagonists.

aPC or activated protein C-refers to recombinant aPC. aPC includes and is preferably recombinant human aPC although aPC may also include other species having protein C proteolytic, amidolytic, esterolytic, and biological (anti-coagulant, anti-inflammatory, or pro-fibrinolytic) activities.

Human protein C derivative(s) refers to the recombinantly produced derivatives of this invention that differ from wild-type human protein C but when activated retain the essential properties i.e., proteolytic, amidolytic, esterolytic, and biological (anti-coagulant, anti-inflammatory, pro-fibrinolytic activities). The definition of human protein C derivatives as used herein also includes the activated form of the above-identified human protein C derivatives.

Treating—describes the management and care of a patient for the purpose of combating a disease, condition, or disorder whether to eliminate the disease, condition, or disorder, or prophylactically to prevent the onset of the symptoms or complications of the disease, condition, or disorder.

Continuous infusion—continuing substantially uninterrupted the introduction of a solution or suspension into a vein for a specified period of time.

Bolus injection—the injection of a drug in a defined quantity (called a bolus) over a period of time up to about 120 minutes.

Suitable for administration—a lyophilized formulation or solution that is appropriate to be given as a therapeutic agent.

Unit dosage form—refers to physically discrete units suitable as unitary dosages for human subjects, each unit containing a predetermined quantity of active material calculated to produce the desired therapeutic effect, in association with a suitable-pharmaceutical excipient.

Hypercoagulable states—excessive coagulability associated with disseminated intravascular coagulation, pre-thrombotic conditions, activation of coagulation, or congenital or acquired deficiency of clotting factors such as aPC.

Zymogen—protein C zymogen, as used herein, refers to secreted, inactive forms, whether one chain or two chains, of protein C.

Protein C deficiency—protein C deficiency as used herein can be congenital or acquired. For either type, the protein C level in circulation is below the lower limit of the normal range. Skilled artisans realize that the normal range is established by a standard protocol utilizing FDA approved equipment and diagnostic kits for determining protein C levels.

Pharmaceutically effective amount—a therapeutically efficacious amount of a pharmaceutical compound. The particular dose of the compound administered according to this invention will, of course, be determined by the attending physician evaluating the particular circumstances surrounding the case, including the compound administered, the particular condition being treated, the patient characteristics and similar considerations.

Acute coronary syndromes—clinical manifestations of coronary atherosclerosis complicated by coronary plaque rupture, superimposed coronary thrombosis, and jeopardized coronary blood flow resulting in coronary ischemia and/or myocardial infarction. The spectrum of acute coronary syndromes includes unstable angina, non-Q-wave (i.e., non-ST-segment elevation) myocardial infarction, and Q-wave (i.e., ST-segment elevation) myocardial infarction.

Thrombotic disorders—a disorder relating to, or affected with the formation or presence of a blood clot within a blood vessel. Such disorders include, but are not limited to, stroke, abrupt closure following angioplasty or stent placement, and thrombosis as a result of peripheral vascular surgery.

Purpura fulminans—ecchymotic skin-lesions, fever, hypotension associated with bacterial sepsis, viral, bacterial or protozoan infections. Disseminated intravascular coagulation is usually present.

Wild-type protein C—the type of protein C that predominates in a natural population of humans in contrast to that of natural or laboratory mutant polypeptide forms of protein C.

Gene Therapy—A therapeutic regime which includes the administration of a vector containing DNA encoding a therapeutic protein, directly to affected cells where the therapeutic protein will be produced. Target tissue for gene delivery include, for example, skeletal muscle, vascular smooth muscle, and liver. Vectors include, for example, plasmid DNA, liposomes, protein-DNA conjugates, and vectors based on adenovirus or herpes virus. Gene therapy has been described, for example, by Kessler et al., PNAS, USA, 93:14082-87, 1996.

Bactericidal permeability increasing protein—includes naturally and recombinantly produced bactericidal permeability increasing (BPI) protein; natural, synthetic, and recombinant biologically active polypeptide fragments of BPI protein; biologically active polypeptide variants of BPI protein or fragments thereof, including hybrid fusion proteins and dimers; biologically active variant analogs of BPI protein or fragments or variants thereof, including cysteine-substituted analogs; and BPI-derived peptides. The complete amino acid sequence of human BPI, as well as the nucleotide sequence of DNA encoding BPI have been elucidated by Gray, et al., 1989, J. Biol. Chem 264:9505. Recombinant genes encoding and methods for expression of BPI proteins, including BPI holoprotein and fragments of BPI are disclosed in U.S. Pat. No. 5,198,541, herein incorporated by reference.

The amino acid abbreviations are accepted by the United States Patent and Trademark Office as set forth in 37 C.F.R. 1.822 (d)(1) (1998).

The present invention provides human protein C derivatives, including activated forms thereof, which have increased anti-coagulant activity as compared to wild-type protein C. The activated form of aPC or human activated protein C derivatives may be produced by activating recombinant human protein C zymogen or recombinant human protein C derivative zymogen in vitro or by direct secretion of the activated form of protein C. The means by which the activation occurs is not critical and the process aspects of this invention include any and all means of activation. Human protein C derivatives may be produced in eukaryotic cells, transgenic animals, or transgenic plants, including, for example, secretion from human kidney 293 cells or AV12 cells as a zymogen, then purified and activated by techniques known to the skilled artisan.

Preferred human protein C derivatives include S12K, S12N, S12H, S11G:S12K, H10Q:S11G:S12K, and H10Q:S11G:S12N and activated forms thereof.

Human protein C derivative S12K preferably contains a lysine residue at position 12 rather than a serine residue normally found at this position; human protein C derivative S12N preferably contains a asparagine residue at position 12 rather than a serine residue normally found at this position; and, human protein C derivative S12H preferably contains a histidine residue at position 12 rather than a serine residue normally found at this position. The activated form of human protein C derivatives S12N and S12H demonstrate increased anti-coagulant activity compared to wild-type aPC, Table 1. It is apparent to one with skill in the art that other amino acid substitutions at residue 12 in addition to lysine and asparagine may impart increased anti-coagulant activity in the resulting derivative molecule. Examples of such amino acid substitutions include Ala, Arg, Asp, Cys, Glu, Gln, Gly, His, Ile, Leu, Met, Phe, Pro, Trp, Thr, Tyr, and Val.

Human protein C derivative S11G:S12K contains a glycine residue at position 11 instead of the serine residue normally found at this position and a lysine at position 12 instead of the serine residue normally found at this position. It is apparent to one with skill in the art that other amino acid substitutions at residue 11 in addition to glycine and at position 12 in addition to lysine may impart increased anti-coagulant activity in the resulting derivative molecule. Examples of such amino acid substitutions include Ala, Arg, Asn, Asp, Cys, Glu, Gln, His, Ile, Leu, Met, Phe, Pro, Thr, Trp, Tyr, and Val, provided that amino acid 10 is not His and amino acid 12 is not Ser.

Human protein C derivative H10Q:S11G:S12K contains a glutamine residue at position 10 instead of a histidine residue normally found at this position, a glycine residue at position 11 instead of the serine residue normally found at this position and a lysine at position 12 instead of the serine residue normally found at this position. The activated form of human protein C derivative H10Q:S11G:S12K demonstrates increased anti-coagulant activity compared to wild-type aPC, Table 1. It is apparent to one with skill in the art that other amino acid substitutions at residue 10 in addition to glycine, at position 11 in addition to glycine, and at position 12 in addition to lysine may impart increased anti-coagulant activity in the resulting derivative molecule. Examples of such amino acid substitutions include Ala, Arg, Asn, Asp, Cys, His, Ser, Lys, Gly, Glu, Gln, Ile, Leu, Met, Phe, Pro, Thr, Trp, Tyr, and Val, provided that amino acid 10 is not His, amino acid 11 is not Ser, and amino acid 12 is not Ser.

Human protein C derivative H10Q:S11G:S12N contains a glutamine residue at position 10 instead of the histidine residue normally found at this position, a glycine residue at position 11 instead of the serine residue normally found at this position and an asparagine residue at position 12 instead of the serine residue normally found at this position. The activated form of human protein C derivative H10Q:S11G:S12N demonstrates increased anti-coagulant activity compared to wild-type aPC, Table 1. It is apparent to one with skill in the art that other amino acid substitutions at residue 10 in addition to glycine, at position 11 in addition to glycine, and at position 12 in addition to asparagine may impart increased anti-coagulant activity in the resulting derivative molecule. Examples of such amino acid substitutions include Ala, Arg, Asp, Cys, Glu, His, Gly, Ser, Gln, Ile, Lys, Leu, Met, Phe, Pro, Thr, Trp, Tyr and Val, provided that amino acid 10 is not His, amino acid 11 is not Ser, and amino acid 12 is not Ser.

Further embodiments of the present invention include human protein C derivatives: S11G:S12H, S11G:S12N, and H10Q:S11G:S12H, and activated forms thereof which have increased anti-coagulant activity as compared to wild-type activated protein C.

Human protein C derivative S11G:S12H contains a glycine residue at position 11 instead of the serine residue normally found at this position and a histidine residue at position 12 instead of the serine residue normally found at this position. The activated form of human protein C derivative S11G:S12H demonstrates increased anti-coagulant activity compared to wild-type aPC, Table 1. It is apparent to one with skill in the art that other amino acid substitutions at residue 11 in addition to glycine and at position 12 in addition to histidine may impart increased anti-coagulant activity in the resulting derivative molecule. Examples of such amino acid substitutions include Ala, Arg, Asn, Asp, Cys, Glu, Gln, Gly, His, Ser, Tyr, Thr, Ile, Leu, Lys, Met, Phe, Pro, Trp, and Val provided that amino acid 10 is not His and amino acid 12 is not Ser.

Human protein C derivative S11G:S12N contains a glycine residue at position 11 instead of the serine residue normally found at this position and an asparagine residue at position 12 instead of the serine residue normally found at this position. The activated form of human protein C derivative S11G:S12N demonstrates increased anti-coagulant activity compared to wild-type aPC, Table 1. It is apparent to one with skill in the art that other amino acid substitutions at residue 11 in addition to glycine and at position 12 in addition to asparagine may impart increased anti-coagulant activity in the resulting derivative molecule. Examples of such amino acid substitutions include Ala, Arg, Asn, Asp, Cys, Glu, Gln, Gly, His, Tyr, Thr, Ile, Leu, Lys, Met, Phe, Pro, Trp, Thr, Tyr, and Val.

Human protein C derivative H10Q:S11G:S12H contains a glutamine residue at position 10 instead of the histidine residue normally found at this position, a glycine residue at position 11 instead of the serine residue normally found at this position and a histidine residue at position 12 instead of the serine residue normally found at this position. It is apparent to one with skill in the art that other amino acid substitutions at residue 10 in addition to glycine, at position 11 in addition to glycine, and at position 12 in addition to histidine may impart increased anti-coagulant activity in the resulting derivative molecule. Examples of such amino acid substitutions include Ala, Arg, Asn, Asp, Cys, Glu, His, Gly, Ser, Gln, Ile, Lys, Leu, Met, Phe, Pro, Thr, Trp, Tyr, and Val provided that amino acid 10 is not His, amino acid 11 is not Ser, and amino acid 12 is not Ser.

In addition, human protein C derivatives may include proteins that represent functionally equivalent gene products. Such an equivalent human protein C derivative may contain deletions, additions, or substitutions of amino acid residues within the amino acid sequence encoded by the protein C derivative gene sequences described above, but which result in a silent change, thus producing a functionally equivalent human protein C derivative gene product. Amino acid substitutions may be made on the basis of similarity in polarity, charge, solubility, hydrophobicity, hydrophilicity, and/or the amphipathic nature of the residues involved.

Thus, the derivatives of the present invention include derivatives having an amino acid sequence at least identical to that of SEQ ID NOS: 3, 4, 5, 6, 7, and 8 or fragments thereof with at least 90% identity to the corresponding fragment of SEQ ID NOS: 3, 4, 5, 6, 7, and 8. Preferably, all of these derivatives retain the biological activity of human aPC. Preferred derivatives are those that vary from SEQ ID NOS: 3, 4, 5, 6, 7, and 8, by conservative substitutions i.e., those that substitute a residue with another of like characteristics. Typical substitutions are among Ala, Val, Leu and Ile; among Ser and Thr; among the acidic residues Asp and Glu; among Asn and Gln; and among the basic residues Lys and Arg; or aromatic residues Phe and Tyr. Particularly preferred are derivatives in which several, 5-10, 1-5, or 1-2 amino acids are substituted, deleted, or added in any combination.

The invention also provides DNA compounds for use in making the human protein C derivatives. These DNA compounds comprise the coding sequence for the light chain of human protein C zymogen or human protein C derivative zymogen positioned immediately adjacent to, downstream of, and in translational reading frame with the pre-propeptide sequence of human protein C zymogen or human protein C derivative zymogen. The DNA sequences preferably encode the Lys-Arg dipeptide which is processed during maturation of the protein C molecule, the activation peptide and the heavy chain of the human protein C derivative. Thus, the human protein C derivatives of the present invention are variant or mutant polypeptides which contain one or more amino acid(s) which differ from the wild-type protein C sequence identified as SEQ ID NO: 1 (which does not contain the pre-propeptide sequence) or the corresponding wild type amino acid in SEQ ID NO:2 (which contains the pre-propeptide sequence).

Those skilled in the art will recognize that, due to the degeneracy of the genetic code, a variety of DNA compounds can encode the derivatives described above. U.S. Pat. No. 4,775,624, the entire teaching of which is herein incorporated by reference, discloses the wild-type form of the human protein C molecule. The skilled artisan could readily determine which changes in the DNA sequences which could encode the exact derivatives as disclosed herein. The invention is not limited to the specific DNA sequences disclosed. Consequently, the construction described below and in the accompanying Examples for the preferred DNA compounds are merely illustrative and do not limit the scope of the invention.

All of the DNA compounds of the present invention were prepared by the use of site-directed mutagenesis to change particular positions within the human protein C zymogen. The technique for modifying nucleotide sequences by site-directed mutagenesis is well known to those skilled in the art. See e.g., Sambrook, Fritsch & Maniatis, Molecular Cloning: A Laboratory Manual, second Edition (1989).

The human protein C derivatives can be made by techniques well known in the art utilizing eukaryotic cell lines, transgenic animals, or transgenic plants. Skilled artisans will readily understand that appropriate host eukaryotic cell lines include but are not limited to HepG2, LLC-MK₂, CHO-K1, 293, or AV12 cells, examples of which are described in U.S. Pat. No. 5,681,932, herein incorporated by reference. Furthermore, examples of transgenic production of recombinant proteins are described in U.S. Pat. Nos. 5,589,604 and 5,650,503, herein incorporated by reference.

Skilled artisans recognize that a variety of vectors are useful in the expression of a DNA sequence of interest in a eukaryotic host cell. Vectors that are suitable for expression in mammalian cells include, but are not limited to: pGT-h, pGT-d; pCDNA 3.0, pCDNA 3.1, pCDNA 3.1+Zeo, and pCDNA 3.1+Hygro (Invitrogen); and, pIRES/Hygro, and pIRES/neo (Clonetech). The preferred vector of the present invention is pIG3 as described in Example 2.

Other sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e. to maintain the proper reading frame.

To be fully active and operable under the present methods, the human protein C derivatives made by any of these methods must undergo post-translational modifications such as the addition of nine gamma-carboxy-glutamates, the addition of one erythro-beta-hydroxy-Asp (beta-hydroxylation), the addition of four Asn-linked oligosaccharides (glycosylation) and, the removal of the leader sequence (42 amino acid residues). Without such post-translational modifications, the protein C derivatives are not fully functional or are non-functional.

It is known in the art that post-translational modifications of recombinant proteins such as the human protein C derivatives of the present invention may vary depending on which host cell line is utilized for the expression of the recombinant protein. For example, the post-translational modification of gamma-carboxylation, which is essential for the anti-coagulant activity of the human protein C derivatives of the present invention, may be higher, slightly lower, or much lower than plasma derived wild-type protein C gamma-carboxylation, depending on the host cell line used (Yan et al., Bio/Technology 8(7):655-661, 1990). Such differences in gamma-carboxylation provide a basis for the use of site-directed mutagenesis to change particular positions within the human protein C molecule that will result in an increase in anti-coagulant activity.

An embodiment of the present invention is increased production levels and increased specific activity of properly gamma-carboxylated protein C and/or protein C with increased anti-coagulant activity by the inhibition of phosphorylation of the serine residue at position 12 as described in Example 1. This inhibition of phosphorylation can be accomplished by replacing the serine residue at position 12 with a non-phosphorylatable amino acid by site-directed mutagenesis, i.e. an amino acid other than Ser, Tyr, or Thr, or by the inhibition of the kinase responsible for the phosphorylation of the serine residue at position 12 by including a non-toxic kinase inhibitor in the tissue-culture medium used to grow the host cell line. It is known for certain cell types, i.e. CHO-K1, that gamma-carboxylation is limited, and therefore, the amount of functional protein C produced by such cells is limited. This lack of carboxylation may be due to phosphorylation of the serine residue at position 12.

Thus, another embodiment of the present invention is increasing production levels and specific activity of a human protein C derivative with increased anti-coagulant activity compared to wild-type activated protein C produced by the process comprising: transforming a host cell with a vector containing nucleic acid encoding a human protein C derivative; culturing said host cell in a medium appropriate for expression of said human protein C derivative wherein phosphorylation of the serine residue at position 12 of SEQ ID NO: 1 and the corresponding amino acid of SEQ ID NO: 2 is inhibited; isolating said human protein C derivative from the culture medium; and activating said human protein C derivative.

Methods for the activation of zymogen forms of human protein C and human protein C derivatives to activated human protein C and activated human protein C derivatives are old and well known in the art. Human protein C may be activated by thrombin alone, by a thrombin/thrombomodulin complex, by RVV-X, a protease from Russell's Viper venom, by pancreatic trypsin or by other proteolytic enzymes.

Additionally, the present invention further relates to the treatment of acute coronary syndromes comprising myocardial infarction, and unstable angina with aPC derivatives with increased anti-coagulation activity as compared to wild-type aPC.

The recombinant human protein C derivatives of the present invention are also useful for the treatment of thrombotic disorders such as stroke, abrupt closure following angioplasty or stent placement, and thrombosis as a result of peripheral vascular surgery.

The recombinant human protein C derivatives of the present invention are useful for the treatment of vascular occlusive disorders or hypercoagulable states associated with sepsis, disseminated intravascular coagulation, major trauma, major surgery, burns, adult respiratory distress syndrome, transplantations, deep vein thrombosis, heparin-induced thrombocytopenia, sickle cell disease, thalassemia, viral hemorrhagic fever, thrombotic thrombocytopenic purpura, and hemolytic uremic syndrome. In another embodiment, the recombinant human protein C derivatives of the present invention are useful for the treatment of sepsis in combination with bacterial permeability increasing protein. In yet another aspect of this invention the activated human protein C derivatives of the present invention are combined with an anti-platelet agent(s) to treat or prevent various thrombotic disorders.

The recombinant human protein C derivatives of the present invention are useful for the treatment of acute arterial thrombotic occlusion, thromboembolism, or stenosis in coronary, cerebral or peripheral arteries or in vascular grafts, in combination with a thrombolytic agent such as tissue plasminogen activator, streptokinase, and related compounds or analogs thereof.

An additional aspect of the invention comprises treating the diseases and conditions caused or resulting from protein C deficiency as defined herein. This aspect of the invention contemplates any and all modifications to any aPC molecule resulting in increased anti-coagulant activity as compared to wild-type aPC.

Yet another aspect of the invention comprises treating genetically predisposed prothrombotic disorders, such as protein C deficiency and Factor V Leiden mutation, by gene therapy with a recombinant DNA molecule encoding a protein C derivative of the present invention.

The human protein C derivatives can be formulated according to known methods to prepare a pharmaceutical composition comprising as the active agent an aPC derivative and a pharmaceutically acceptable solid or carrier. For example, a desired formulation would be one that is a stable lyophilized product of high purity comprising a bulking agent such as sucrose, trehalose or raffinose; a salt such as sodium chloride or potassium chloride; a buffer such as sodium citrate, Tris acetate, or sodium phosphate, at a pH of about 5.5 to about 6.5; and an activated human protein C derivative. A preferred stable lyophilized formulation comprises a weight ratio of about 1 part activated protein C derivative, between 7 to 8 parts salt and between about 5 and 7 parts bulking agent. Examples of stable lyophilized formulations include: 5.0 mg/ml activated protein C derivative, 30 mg/ml sucrose, 38 mg/ml NaCl and 7.56 mg/vial citrate, pH 6.0; and, 20 mg/vial activated protein C derivative, 120 mg/ml sucrose, 152 mg/vial NaCl, 30.2 mg/vial citrate, pH 6.0.

The amount of human aPC derivative administered will be from about 0.01 μg/kg/hr to about 50 μg/kg/hr. More preferably, the amount of human aPC derivative administered will be about 0.1 μg/kg/hr to about 25 μg/kg/hr. Even more preferably the amount of human aPC derivative administered will be about 1 μg/kg/hr to about 15 μg/kg/hr. The most preferable amounts of human aPC derivative administered will be about 5 μg/kg/hr or about 10 μg/kg/hr.

Preferably, the human aPC derivatives will be administered parenterally to ensure delivery into the bloodstream in an effective form by injecting a dose of about 0.01 μg/kg/hr to about 50 μg/kg/hr, as a continuous infusion for 1 to 240 hours. More preferably, the human aPC derivatives will be administered as a continuous infusion for 1 to 196 hours. Even more preferably, the human aPC derivatives will be administered as a continuous infusion for 1 to 144 hours. Yet even more preferably, the aPC derivatives will be administered as a continuous infusion for 1 to 96 hours.

The plasma ranges obtained from the amount of aPC administered will be 0.02 ng/ml to less than 100 ng/ml. The preferred plasma ranges are from about 0.2 ng/ml to 50 ng/ml. Most preferably plasma ranges are from about 2 ng/ml to about 30 ng/ml and still more preferably about 10 ng/ml to about 20 ng/ml.

Alternatively, the human aPC derivative will be administered by injecting a portion (⅓ to ½) of the appropriate dose per hour as a bolus injection over a time from about 5 minutes to about 120 minutes, followed by continuous infusion of the appropriate dose for up to 240 hours.

In another alternative, the human aPC derivatives will be administered at a dose of 0.01 mg/kg/day to about 1.0 mg/kg/day, B.I.D. (2 times a day), for one to ten days. More preferably, the human aPC derivatives will be administered B.I.D. for three days.

In yet another alternative, the human aPC derivatives will be administered subcutaneously at a dose of 0.01 mg/kg/day to about 1.0 mg/kg/day, to ensure a slower release into the bloodstream. Formulation for subcutaneous preparations will be done using known methods to prepare such pharmaceutical compositions.

Another aspect of the invention is an article of manufacture for human pharmaceutical use, comprising packaging material and a vial comprising lyophilized human activated protein C derivative with increased anti-coagulant activity when compared to wild-type activated protein C, wherein said packaging material comprises a label which indicates that said activated protein C be administered as a continuous infusion at a dosage of about 0.01 μg/kg/hr to about 50 μg/kg/hr, for 1 to 240 hours. Additionally, this aspect of the invention includes other routes of administration such as bolus plus continuous infusion, B.I.D., or subcutaneous injection.

The phrase “in combination with” as used herein, refers to the administration of additional agents with aPC either simultaneously, sequentially or a combination thereof. Examples of additional agents are anti-platelet agents and BPI protein.

The human aPC derivatives described in this invention have essentially the same type of biological activity as the wild-type human aPC, with substantially increased anti-coagulant activity. Therefore, these compounds will require either less frequent administration and/or smaller dosage. Finally, superior increases in human aPC derivative anti-coagulant activity may be achieved via one to three amino acid substitutions, which are less likely to be immunogenic than aPC derivatives with more than three amino acid substitutions.

The following Examples are provided merely to further illustrate the present invention. The scope of the invention shall not be construed as merely consisting of the following Examples.

### EXAMPLE 1

### Identification of Specific Target Residues for Site-Directed Mutagenesis

Human protein C (hPC), expressed in Syrian hamster AV12 cells was analyzed by HPLC/MS, MS/MS and N-terminal sequencing. hPC was reduced, alkylated and deglycosylated with N-glycosidae F or digested with trypsin. HPLC/MS analysis for the reduced, alkylated and deglycosylated sample indicated that the heavy chain molecular weight was consistent with the molecular weight predicted from the amino acid sequence. However, only about 70% of the light chain had the expected molecular weight. The remaining ˜30% had a molecular weight 316 daltons less than the expected value. This reduced molecular weight light chain fraction was collected, treated with trypsin and then analyzed by HPLC/MS, MS/MS and N-terminal sequencing. The results showed that serine residue 12 of the light chain was phosphorylated; additionally, this material did not contain any of the expected gamma-carboxyglutamic acid residues. Thus, hPC containing a light chain with a phosphorylated serine 12 residue will have reduced or no anti-coagulant activity since it lacks gamma-carboxyglutamic acid residues.

This observation suggests that replacement of serine 12, with a non-phosphorylatable residue, as well as changes in the-sequence near serine 12, would either reduce or prevent phosphorylation, and therefore increase the amount of properly gamma-carboxylated protein C. Site-directed mutagenesis was used to explore the effects of changing residues at positions 10, 11, and 12 (both individually, and in combinations) on human protein C derivative anti-coagulant activity.

It is well known that amino acid residues Ser, Thr, and Tyr are readily phosphorylated. Therefore, replacement of the serine residue at position 12 with Ala, Arg, Asn, Asp, Cys, Glu, Gln, Gly, His, Ile, Leu, Lys, Met, Phe, Pro, Trp, or Val results in a non-phosphorylatable residue at this position. Additionally, inhibition of the kinase responsible for the phosphorylation of the serine residue at position 12 via the inclusion of a non-toxic kinase inhibitor into the tissue-culture medium, will prevent phosphorylation of the serine at position 12 and therefore enhance yields of properly gamma-carboxylated protein C.

Thus, either site-directed mutagenesis to change amino acid residues at positions 10, 11, or 12, or the inclusion of a non-toxic kinase inhibitor in the tissue-culture medium, results in the prevention of phosphorylation of the serine residue at position 12. This in turn results in enhanced yields of properly gamma-carboxylated protein C with increased anti-coagulant activity when compare to wild-type aPC.

### EXAMPLE 2

### Protein C Derivative Construction and Production

Human protein C derivatives were constructed using the polymerase chain reaction (PCR) following standard methods. The source of the wild-type coding sequence was plasmid pLPC (Bio/Technology 5:1189-1192, 1987). The universal PCR primers used include: PC001b; 5′-GCGATGTCTAGAccaccATGTGGCAGCTCACAAGCCTCCTGC-3′, which encodes for an XbaI restriction site (underlined) used for subcloning, a Kozak consensus sequence (lowercase) (Kozak, J Cell Biol 108(2):229-41, 1989), and the 5′ end of the coding region for protein C: PC002E; 5′-CAGGGATGATCACTAAGGTGCCCAGCTCTTCTGG-3′, which encodes for the 3′ end of the coding region for human protein C, and includes a BclI restriction site (underlined) for subcloning. All site-directed mutagenesis was accomplished by established PCR methodology, using complementary oligonucleotides containing the desired sequence changes. The first round of PCR was used to amplify two fragments of the protein C gene; the 5′ fragment was generated using PC001b and the antisense mutagenic primer, and the 3′ fragment was generated using PC002e and the sense mutagenic primer. The resulting amplified products were purified by standard procedures. These fragments were combined and then used as a template for a second round of PCR using primers PC001b and PC002e. The final PCR product was digested with XbaI and BclI and subcloned into similarly digested expression vector pIG3. A wild-type construct was similarly generated by PCR using the two universal primers and the plasmid pLPC as the template, followed by subcloning into pIG3. The mutations were confirmed by DNA sequencing of both the coding and non-coding strands. The pIG3 vector was generated by the insertion of an “internal ribosome entry site” (IRES) (Jackson, et al., Trends Biochem Sci 15(12):447-83, 1990) and green fluorescent protein (GFP) (Cormack, et al., Gene 173:33-38, 1996) gene into the mammalian expression vector pGTD (Gerlitz, et al., Biochem J 295(Pt 1):131-40, 1993). When a cDNA of interest is cloned into the multiple cloning site of pIG3, the GBMT promoter (Berg, et al., Nucleic Acids Res 20(20):5485-6, 1992) drives expression of a bicistronic mRNA (5′-cDNA-IRES-GFP-3′). Efficient translation of the first cistron is initiated by classical assembly of ribosome subunits on the 5′-methylated cap structure of the mRNA; while the normally inefficient translation of a second cistron is overcome by the IRES sequence which allows for internal ribosome assembly on the mRNA. The coupling of the cDNA and reporter on a single mRNA, translated as separate proteins, allows one to screen for the highest-producing clones on the basis of fluorescence intensity. The expression vector also contains an ampicillin resistance cassette for maintenance of the plasmid in E. coli, and a murine DHFR gene with appropriate expression sequences for selection and amplification purposes in mammalian tissue expression.

The adenovirus-transformed Syrian hamster AV12-664 cell line was grown in Dulbecco's modified Eagle's medium supplemented with 10% fetal bovine serum, 50 μg/mL gentamicin, 200 μg/mL Geneticin (G418), and 10 μg/mL vitamin K1. One day prior to transfection, cells were plated at a density of about 10⁵ cells/25 cm². FspI-linearized plasmids were transfected using either the calcium phosphate method (ProFection, Gibco BRL-Life Technologies) or FuGene-6 (Boehringer Mannheim), following the manufacturer's instructions. Approximately 48 hours after transfection, the medium was replaced with medium containing 250 nM methotrexate for selection. Colonies resistant to methotrexate were pooled 2-3 weeks after applying drug selection and expanded. The pools were subjected to fluorescence activated cell sorting based upon GFP fluorescence intensity (Cormack, 1996), with the most intense 5% of fluorescent cells being retained and expanded. To obtain material for purification, recombinant cells were grown in a modified mixture of Dulbecco's modified Eagle's and Ham's F-12 media (1:3) containing 1 μg/mL human insulin, 1 μg/mL human transferrin, and 10 μg/mL vitamin K1. Conditioned media were collected, adjusted to a final concentration of 5 mM benzamidine and 5 mM EDTA, pH 8.0, and protein C was purified via anion-exchange chromatography as described (Yan, et al., Bio/Technology 8:655-661, 1990). Purified protein was desalted/concentrated in Ultrafree-CL 30,000 NMWL filtration units (Millipore) using Buffer A (150 mM NaCl, 20 mM Tris-HCl, pH 7.4), and quantitated by Pierce BCA assay using bovine serum albumin (BSA) as the standard.

### EXAMPLE 3

### Activation of Recombinant Protein C

Complete activation of the zymogen forms of protein C and protein C derivatives was accomplished by incubation with thrombin-sepharose. Thrombin-sepharose was washed extensively with Buffer A. 50 μL of packed thrombin-sepharose was mixed with 250 μg of protein C in 1 mL of the same buffer and incubated at 37° C. for 4 hours with gentle shaking on a rotating platform. During the course of the incubation, the degree of protein C activation was monitored by briefly pelleting the thrombin-sepharose, and assaying a small aliquot of the supernatant for aPC activity using the chromogenic substrate S-2366 (DiaPharma). Following complete activation, the thrombin-sepharose was pelleted, and the supernatant collected. aPC concentration was verified by Pierce BCA assay, and the aPC was either assayed directly, or frozen in aliquots at −80° C. All derivatives were analyzed by SDS-PAGE with either Coomassie-blue staining or Western Blot analysis to confirm complete activation (Laemmli, Nature 227:680-685, 1970).

### EXAMPLE 4

### Functional Characterization

The amidolytic activity of recombinant human aPC derivatives was determined by hydrolysis of the tri-peptide substrates S-2366 (Glu-Pro-Arg-p-nitroanilide), S-2238 (Pip-Pro-Arg-p-nitroanilide), and S-2288 (Ile-Pro-Arg-p-nitroanilide). The anti-coagulant activity is shown as measured clotting time in an aPTT at 500 ng mL⁻¹ aPC. Amidolytic activities were measured using the chromogenic substrate S-2366.

Assays were performed at 25° C., in Buffer A containing 1 mg mL⁻¹ BSA, 3 mM CaCl₂, and 0.5 nM aPC. Reactions (200 μL/well) were performed in a 96-well microtiter plate, and amidolytic activity was measured as the change in absorbance-units/min at 405 nm as monitored in a ThermoMax kinetic micrometer plate reader. Kinetic constants were derived by fitting velocity data at varying substrate concentrations (16 μM to 2 mM) to the Michaelis-Menten equation. Changes in A₄₀₅ were converted to mmol product using a path length of 0.53 cm (Molecular Devices Technical Applications Bulletin 4-1), and an extinction coefficient for the released p-nitroanilide of 9620 M⁻¹ cm⁻¹ (Pfleiderer, Methods Enzymol 19:514-521, 1970).

Anti-coagulant activity was assessed by measuring the prolongation of clotting time in the activated partial thromboplastin time clotting assay (Helena Laboratories). Clotting reactions were monitored in a ThermoMax kinetic microtiter plate reader, measuring the time to Vₘₐₓ in the change in turbidity. Relative anti-coagulant activities of gla-domain mutant human protein C derivatives are shown in Table 1. Values are based upon the concentration required to double the APTT time, relative to wild-type aPC.

TABLE 1
Relative anti-coagulant activities of gla-domain
variants.

| Variant | Relative aPTT activity |
|---|---|
| S12N | 2.5 X |
| S12H | 2 X |
| S11G/S12N | 1.5 X |
| S11G/S12H | 1.5 X |
| H10Q/S11G/S12N | 3 X |
| H10Q/S11G/S12K | 4 X |

1-76. (canceled)

77. A human protein C derivative consisting of an amino acid sequence selected from the group consisting of SEQ ID NO: 1 derivatives S12H, S12N, S11G:S12H, S11G:S12N, H10Q:S11G:S12K, and H10Q:S11G:S12N.

78. The human protein C derivative of claim 77, wherein said human protein C derivative is in its activated form.

79. A pharmaceutical composition comprising: the human protein C derivative of claim 77 in a pharmaceutically acceptable diluent.

80. The human protein C derivative of claim 77 produced by a process comprising:
(a) transforming a host cell with a vector containing nucleic acid encoding the human protein C derivative of claim 77;
(b) culturing said host cell in a medium appropriate for expression of said human protein C derivative;
(c) isolating said human protein C derivative from the culture medium; and
(d) activating said human protein C derivative.
